(19) United States
(12) Patent Application Publication
(10) Pub. No.: US 2004/0251137 A1
(43) Pub. Date: Dec. 16, 2004

(54) Slope electrophoresis

(75) Inventors: John H. Lee, Olathe, KS (US); Jane Q. Lee, Olathe, KS (US)

Correspondence Address: RIGEL TECHNOLOGY CORPORATION, 11800 WEST 63 STREET, SUITE 26, SHAWNEE, KS, 66203, US

(73) Assignee: Rigel Technology Corporation, Shawnee, KS (US)

(21) Appl. No.: 10/457,327

(22) Filed: Jun. 10, 2003

(51) Int. Cl. C25B 7/00; G01N 33/559; B01D 57/02

(52) U.S. Cl. 204/466; 204/467

(57) ABSTRACT

New slope electrophoresis method and apparatus for performing electrophoresis as a biochemical analysis tool is disclosed. Slope electrophoresis combines the advantages of vertical electrophoresis and horizontal electrophoresis. The angle of slope electrophoresis to the bottom side is between horizontal electrophoresis (0 degree) and vertical electrophoresis (90 degree). The most prefer angle of slope of electrophoresis is between 75 degree to 88 degree. Slope electrophoresis is also used for two-dimensional electrophoresis.

### FIELD OF THE INVENTION

The present invention relates to new slope electrophoresis for performing electrophoresis as a new biochemical analysis tool. Slope electrophoresis combines the advantages of vertical electrophoresis and horizontal electrophoresis.

### BACKGROUND OF THE INVENTION

Vertical electrophoresis and horizontal electrophoresis have been used for separating and analyzing proteins, DNA or RNA fragments and other biomolecular components. Vertical electrophoresis has an angle 90 degree to the bottom side, which helps to increase the separation resolution by an electrical field and major down-direction diffusion. The gel slab of vertical electrophoresis composes of two flat glass plates separated by thin strips placed at opposite edges and between these plates enclosing a gel composed of such as polyacrylarnide or agarose cast between the plates. The electrophoresis separation is carried out within the gel between the two glass plates. The gel slab with the two flat glass plates is secured to a vertical support platform to avoid the leakage problem during electrophoresis operation. There are two reservoirs for containing buffers, one installed toward the upper end of the vertical slab support and a second installed toward the lower end of the vertical slab support. Then a voltage is applied between the upper and lower end buffers added to the reservoirs. An effective electrical contact is established between the buffers in the two reservoirs through the gel to be used to electrophoretically separate the bimolecular components of the test samples. Horizontal electrophoresis has an angle 0 degree to the bottom side or they are parallel each other. The two buffer reservoirs are at the same level. The gel slab composes of such as polyacrylamide or agarose gel cast on a plastic film as a support, which makes the gel slab to be handled easily. The gel slab with the film support is laid onto horizontal electrophoresis apparatus. Two edges of the gel slab contact into the buffers of the two buffer reservoirs. Besides the biomolecular components move to one direction under an electrical field at different speed. Also the components diffuse at all directions because of the horizontal level, which is the major reason to make horizontal electrophoresis at lower separation resolution compared with the separation resolution of vertical electrophoresis.

Vertical electrophoresis has higher separation resolution. Also vertical electrophoresis is more complex to be handled. There is the buffer leakage problem if the glass plates are not hold well. Also the electrophoresis gel is usually difficult to be dried without breaking into pieces. Horizontal electrophoresis is easy to be handled. Also horizontal electrophoresis usually has no problems such as the buffer leakage and broken gel.

Over the years, various attempts have been made to relate and to improve vertical electrophoresis and horizontal electrophoresis systems. A number of patents have been issued for these attempts. U.S. patent application Ser. No. 29/163,385, which is in the issue process, discloses a new design of new slope electrophoresis by the authors in this present invention. U.S. Pat. No. 6,527,933 discloses a new DNA sample preparation and electrophoresis analysis apparatus. U.S. Pat. No. 6,562,213 discloses an electrophoresis-apparatus for simultaneous loading of multiple samples for molecular separation. U.S. Pat. No. 6,507,664 discloses an automated system for two-dimensional electrophoresis to separate, identify and characterize biomolecules. U.S. Pat. No. 6,451,193 discloses an electrophoresis cell for multiple slab gels. U.S. Pat. No. 6,420,072 discloses a process method to prepare a crosslinked polymer gel, which is excellent in heat resistance and durability in electroconductivity. U.S. Pat. No. 6,203,679 discloses an electrophoresis gel container apparatus, which transmits UV light. The gel can be used and analyzed. U.S. Pat. No. 5,885,433 discloses a heat sink plate of an electrophoresis unit. A face-to-face contact is uniform across the plates and hence, heat transfer from the gel slab maintained uniform. U.S. Pat. No. 5,637,203 discloses a platform for conducting electrophoresis and electrophoresis plate for use with the platform. U.S. Pat. No. D346,662 discloses a combined stacked temperature regulator, power supply and gel dryer for electrophoresis. U.S. Pat. No. D315,952 discloses a ornamental design for a horizontal electrophoresis unit. U.S. Pat. No. D303,007 discloses a design for an electrophoresis sequencing apparatus. U.S. Pat. No. 4,773,984 discloses a vertical gel slab electrophoresis apparatus. Both upper and lower buffer reservoirs are provided with hinged covers which act as electrical interlocks to prevent unsafe operation of the apparatus, as well as prevent spillage of buffer solution. U.S. Pat. No. D269,123 discloses a special design for an electrophoresis apparatus.

### SUMMARY OF THE INVENTION

The present invention provides new method and apparatus of slope electrophoresis, which overcomes the problems of horizontal electrophoresis and vertical electrophoresis and combines the high separation resolution of vertical electrophoresis and easy handling of horizontal electrophoresis. The various novel elements in the present invention are disclosed.

The angle of slope electrophoresis to the bottom side is between horizontal electrophoresis (0 degree) and vertical electrophoresis (90 degree). The prefer angle of slope of electrophoresis is between 45 degree to 90 degree. The most prefer angle of slope electrophoresis is between 75 degree to 88 degree. Proteins, DNA or RNA fragments and other components are separated under an electrical field. Also the components diffuse to a major down direction when the slope is close to vertical angle of vertical electrophoresis. The separation resolution by slope electrophoresis is improved significantly compared with horizontal electrophoresis.

Even our design patent application for the new idea and apparatus of slope electrophoresis has been granted as an US patent, which is in the issue process, our experiments show that it is necessary to specify more details for the new idea, method, apparatus and related products, which are only covered by this utility patent application in the present invention. A trademark application of “slope electrophoresis” is in the process.

### DETAILED DESCRIPTION

The gel slab and connector formations and slope electrophoresis apparatus of the present invention provide advantages and capabilities heretofore unavailable in horizontal electrophoresis and vertical electrophoresis.

There are upper and lower buffer reservoirs at different levels in slope electrophoresis, which provide a slope for the gel slab of slope electrophoresis. The positions of the upper and lower buffer reservoirs can be fixed or adjustable to fit the fixed or different length of the gel slab. The gel slab composes of such as polyacrylamide or agarose gel cast onto a plastic film with hydrophilic side up or plastic or glass plate or the combination of the plastic film and plastic or glass plate as a support, which makes the gel slab to be handled easily. The combination of the gel slab with the plastic film and plastic or glass plate makes the gel slab to have a hard surface. The gel slab with the plastic film helps to avoid the electrophoresis gel to be broken easily during the dry process, which is similar with the gel slab with the plastic film of horizontal electrophoresis. The gel with the test samples is attached onto the plastic film by an air dryer. The lower end of the gel slab of slope electrophoresis contacts with lower buffer. The upper end of the gel slab of slope electrophoresis contacts with upper buffer in two ways. One way is the top part of the gel slab to have some angle structure, which expands the gel slab and to contact with upper buffer by the end of the top part of the gel slab. Another way is to use a gel connector with some angle structure to connect upper buffer and the flat gel slab by such as polyacrylamide or agarose gel. There are two pieces of the gel slab and the gel connector. The first way is to make the two pieces into one piece, which usually has more advantages. Test liquid samples are loaded into the sample holes on the gel slab. Then a voltage is applied between the upper and lower end buffers added to the reservoirs. An effective electrical contact is established between the buffers in the two reservoirs through the gel to be used to electrophoretically separate the biomolecular components of the test samples. The components move to one direction under an electrical field at different speed such as according to different molecule weight, charge or electrofocusing. Also the components diffuse at a major down direction because of the slope level, which is the major reason to make slope electrophoresis with higher separation resolution compared with horizontal electrophoresis. If the gel slab and the connector together as one piece, the gel connector part is cut after the operation and the gel part with the plastic film is processed as electrophoresis gel for such as press, dry, stain, destain and dry treatments.

Current gel slab of horizontal electrophoresis or vertical electrophoresis can also be used in slope electrophoresis with the gel connector, which connects between upper buffer and the top end of the gel slab of horizontal electrophoresis or vertical electrophoresis. If the gel slab of vertical electrophoresis with two flat glass plates and thin strips is used in slope electrophoresis, the upper buffer reservoir chamber may be cut to be open to make the gel slab and upper buffer to contact each other directly, which is similar to the vertical electrophoresis for nucleic acid analysis. The three sides of the open chamber of the upper buffer reservoir are attached with such as some rubber strips, which helps the gel slab with the flat glass plates and thin strips to be hold firmly with the upper buffer chamber and the support of the gal slab to avoid the buffer leakage during the operation. The open number may be from one to four for loading one to four gel slabs during electrophoresis operation. The gel slab can be hold firmly by some holders or locks such as screws or clumps for the gel slab of vertical electrophoresis or slop electrophoresis. If the distances of height and intercept of the two buffer reservoirs in slope electrophoresis apparatus are fixed, the apparatus fits to a certain length of the gel slab. If the distances of height of the two buffer reservoirs are adjustable by such as screws or clumps, then the slope electrophoresis apparatus fits to different length of the gel slab, which is more flexible. It is better to fit different wideness of the gel slab. Also removable buffer reservoirs let users to easily discard the used buffer after the operation.

Horizontal electrophoresis is usually used to run one gel slab. Vertical electrophoresis is usually used to run two gel slabs at the same time. Slope electrophoresis is usually used to run one or two gel slabs at the same time. Also slope electrophoresis can be used to run three or four gel slabs at the same time, which depends on the apparatus structure. Heat is produced during electrophoresis operation, which raises the temperature of the gel slab. A support sheet or box with cooling or tap water to the surface of the gel slab can be used to cool the temperature of the gel slab to avoid the overheat. Some cover to slope electrophoresis apparatus or parts is used for the safety during electrophoresis operation. After the electrophoresis operation is done, the power is turned off and the cover is removed. Then the gel slab is processed for such as press, dry, stain, destain and dry processes. After running one sample in the gel slab of slope electrophoresis and then turning the gel slab to 90 degree, two-dimensional electrophoresis can be done easily by slope electrophoresis. Two-dimensional electrophoresis needs to use capillary electrophoresis apparatus at first. Then vertical electrophoresis apparatus is used for the second dimension. Slope electrophoresis is a perfect tool to be used for two-dimensional electrophoresis, which is much more convenient and economical.

Vertical electrophoresis can also be improved with above methods in two situations. The first is to use the gel connector with some angle structure to connect upper buffer and the top end of the normal gel slab of vertical electrophoresis or the gel slab with a plastic film, two glass plates and thin strips to avoid to avoid the electrophoresis gel to be broken easily during dry process, which changes the direct contact between the gel slab and upper buffer in vertical electrophoresis into the indirect contact by the gel connector. After the electrophoresis operation, the gel with the plastic film is removed easily from the glass plate. Then the gel with the plastic film is processed such as press, dry, stain, destain and dry treatments. The additional plastic film avoids to break the gel easily during dry treatment, which is very common for the normal gels in vertical electrophoresis. The second is to use the gel slab with the gel connector in one-piece form, which is similar to the one gel slab form of slope electrophoresis. The top part has some angle structure. The gel and process can be handled easily in the second situation.

What is claimed is:

1. A method and apparatus of preparing slope electrophoresis comprising a gel slab between lower and upper buffer reservoirs at a slope angle to form the slope electrophoresis with electrophoresis parts.

2. The method of claim 1 wherein the angle of the gel slab of the slope electrophoresis to the bottom side is between horizontal electrophoresis (0 degree) and vertical electrophoresis (90 degree). The prefer angle of the slope of electrophoresis is between 45 degree to 90 degree. The most prefer angle of the slope electrophoresis is between 75 degree to 88 degree.

3. The method of claim 1 wherein the slope electrophoresis apparatus is fixed or adjustable by screws or clamps to fit the lengths of the gel slabs.

4. The method of claim 1 wherein the slope electrophoresis is used to run one to four gel slabs.

5. The method of claim 1 wherein the slope electrophoresis is used to run one-dimensional electrophoresis or two-dimensional electrophoresis.

6. The method of claim 1 wherein the gel slab of the slope electrophoresis is with a gel connector together as one piece and contacts the upper and lower buffers by the two ends.

7. The method of claim 1 wherein a gel connector contacts the gel slab of the slope electrophoresis at one end and the upper buffer at another end.

8. The method of claim 1 wherein a gel connector contacts a gel slab of horizontal electrophoresis at one end and the upper buffer at another end.

9. The method of claim 1 wherein the gel is supported by a plastic film or plastic or glass plate.

10. The method of claim 1 wherein a support box to the gel slab surface is filled with cooling water to control the temperature of the gel slab during the electrophoresis operation to avoid the overheat.

11. The method of claim 1 wherein screws on the bottom are used to adjust to the horizontal level.

12. The method of claim 1 wherein the gel slab contacts the upper buffer directly through an open side of upper buffer reservoir and the gel slab with the glass plates is hold firmly with the outside surface of the upper buffer reservoir by screws or clumps.

13. The method of claim 1 wherein the gel slab, gel connector and electrophoresis apparatus products of slope electrophoresis are made.

14. A method and apparatus of preparing a vertical electrophoresis comprising a gel connector with some angle structure contacts a gel slab at one end and the upper buffer at another end.

15. The method of claim 14 wherein the gel connector with some angle structure is together or separately with the gel slab.

16. The method of claim 14 wherein an adjustable apparatus of the vertical electrophoresis to fit different lengths of the gel slabs.

17. The method of claim 14 wherein the gel connector, the gel slab with the gel connector and the electrophoresis apparatus with the adjustable function are made.

18. A method of preparing a gel slab of vertical electrophoresis comprising a gel slab with an electrophoresis gel, plastic film, two glass plates and thin strips to avoid the electrophoresis gel to be broken easily during the dry process.

19. The method of claim 18 wherein the gel products of vertical electrophoresis are made.
